(19) United States
(12) Patent Application Publication
(10) Pub. No.: US 2021/0371800 A1
(43) Pub. Date: Dec. 2, 2021

(54) SYSTEMS AND METHODS FOR THERMAL MANAGEMENT OF ALGAE CULTIVATION SYSTEMS

(71) Applicant: ExxonMobil Research and Engineering Company, Annandale, NJ (US)

(72) Inventors: Gregory R. Johnson, Annandale, NJ (US); Everett J. O'Neal, Asbury, NJ (US); Sarah E. Feicht, Raritan, NJ (US)

(21) Appl. No.: 17/190,515

(22) Filed: Mar. 3, 2021

Related U.S. Application Data
(60) Provisional application No. 63/032,769, filed on Jun. 1, 2020.

(51) Int. Cl. C12M 1/00; C12M 1/12; C12N 1/12

(52) U.S. Cl. CPC C12M 41/14; C12N 1/12; C12M 23/06

(57) ABSTRACT

Systems and methods for algae cultivation and, more particularly, systems and methods for controlling algae cultivation water slurry temperature to optimize algae facility production and facilitation of algae facility commercial deployment in a wide spectrum of environmental locations. Thermal reservoirs comprising temperature-based, phase-transitioning material(s) are integrated with algae cultivation vessels to provide temperature control of cultivating algae slurries.

### CROSS-REFERENCE TO RELATED APPLICATIONS

This application is related to and claims the benefit of priority from U.S. Provisional Application No. 63/032,769 filed Jun. 1, 2020, which is hereby incorporated by reference in its entirety.

### FIELD OF THE INVENTION

This application relates to systems and methods for algae cultivation.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources.

Algae are photoautotrophic organisms that can survive, grow, and reproduce with energy derived from the sun through the process of photosynthesis. Photosynthesis is a carbon recycling process through which inorganic CO₂ combines with solar energy, other nutrients, and cellular biochemical processes to output gaseous oxygen and to synthesize carbohydrates and other compounds critical to the life of the algae.

To produce algal biomass, algae cells are generally grown in a water slurry comprising water and nutrients. The algae may be cultivated in indoor or outdoor environments, and in closed or open cultivation systems. Closed cultivation systems include photobioreactors, which utilize natural or artificial light to grow algae in an environment that is generally isolated from the external atmosphere. Such photobioreactors may be in a variety of shaped configurations, but are typically tubular or flat paneled. Open cultivation systems include natural and artificial ponds that utilize sunlight to facilitate photosynthesis. Artificial ponds are often shaped in circular or raceway-shaped (oval) configurations.

Various processing methods exist for harvesting cultivated algal biomass to extract lipids therefrom for the production of fuel and other oil-based products. Moreover, harvesting cultivated algal biomass can be used to produce non-fuel or non-oil-based products, including nutraceuticals, pharmaceuticals, cosmetics, chemicals (e.g., paints, dyes, and colorants), fertilizer and animal feed, and the like. However, algae growth and robustness depends on, among other things, the stability of the temperature of the water slurry during cultivation. Even minor fluctuations in water slurry temperatures may hinder and/or be lethal to algae growth and, therefore, delay or otherwise impede algal biomass product production.

Because algal biomass produces valuable commodities, including sustainable biofuels and non-oil based products, control of algae water slurry temperature fluctuations that may affect the quality and/or quantity of the biomass and downstream resultant products is desirable.

### SUMMARY OF THE INVENTION

This application relates to systems and methods for algae cultivation and, more particularly, to systems and methods for controlling algae cultivation water slurry temperature to optimize algae facility production and facilitation of algae facility commercial deployment in a wide spectrum of environmental locations.

In one or more aspects, the present disclosure provides a system comprising a cultivation vessel for containing an algae water slurry for cultivation. A thermal reservoir is integrated with the cultivation vessel for contact with and temperature control of the algae water slurry, the thermal reservoir comprising a temperature-based, phase-transitioning material.

In one or more aspects, the present disclosure provides a method of cultivating an algae water slurry within a cultivation vessel, and controlling a temperature of the algae water slurry using a thermal reservoir integrated with the cultivation vessel and in contact with the algae water slurry, the thermal reservoir comprising a temperature-based, phase-transitioning material.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 is a plot showing an example temperature trend of an actual algae water slurry as compared to actual ambient temperature during algal biomass cultivation.

FIG. 2 is a schematic illustration of an isometric view of a portion of an integrated system comprising an open cultivation and thermal reservoir.

FIG. 3 is a schematic illustration of a side view of a portion of an integrated system comprising a closed cultivation and thermal reservoir.

FIG. 4 is a schematic illustration of a side view of a portion of an integrated system comprising a closed cultivation and thermal reservoir.

### DETAILED DESCRIPTION OF THE INVENTION

This application relates to systems and methods for algae cultivation and, more particularly, to systems and methods for controlling algae cultivation water slurry temperature to optimize algae facility production and facilitation of algae facility commercial deployment in a wide spectrum of environmental locations.

Biofuel production from cultivated algae slurries offers sustainable energy solutions to reduce reliance on fossil fuels and reduce greenhouse gas emissions. Other non-oil-based products can additionally be derived from algal biomass, including nutraceuticals, pharmaceuticals, cosmetics, chemicals (e.g., paints, dyes, and colorants), fertilizer and animal feed, and the like. To accomplish substantial economic, environmental, and societal impact, cultivated algae must be of sufficient quality and in sufficient quantity for harvesting and processing. Moreover, production of healthy algal biomass at reduced energy and operational expenditures allows algae-derived fuels and other non-oil-based products to become more cost-effective for production by algae facilities and, thus, more widely available to the public.

Algae cells may be cultivated in algae slurries that can be upwards of hundreds to thousands of liters or more in volume, depending on the type and configuration of the particular cultivation system. Algae cells are generally cultivated for a predetermined period of time and allowed to reach a particular desired concentration in the algae slurry before the algal biomass is harvested. Depending on the type and configuration of the particular cultivation system, the predetermined cultivation time may be about one day to about one or more weeks. Accordingly, the amount of algal biomass for harvesting may be relatively dilute compared to the total volume of the algae slurry. Because algae growth and robustness depends critically on the temperature of the algae slurry during cultivation, certain cultivation temperatures may be too high or too low, thereby hindering or being lethal to the production of quality algal biomass. Moreover, the majority of algae cultivation systems are located in outdoor algae facilities (whether in open or closed cultivation vessels), whereby daytime and nighttime conditions, as well as other weather phenomena (e.g., wind conditions) exacerbate algae slurry temperature fluctuations. Indeed, algae facilities are typically located in non-agricultural lands that are exposed to high solar intensity (having little to no foliage ground coverage or over coverage), such as arid or desert lands, with relatively high maximum daytime temperatures (e.g., about 35° C. to 40° C., or higher) and relatively low minimum nighttime temperatures (e.g., about 0° C. to 15° C., or lower).

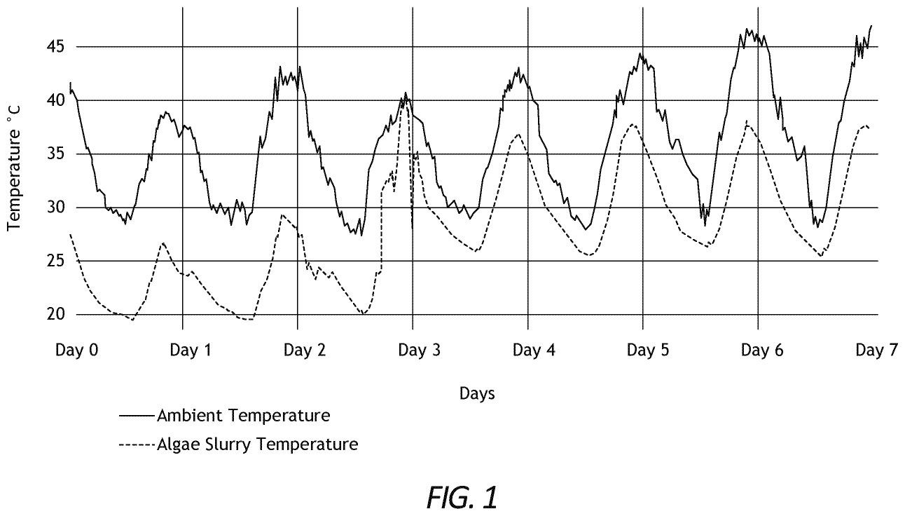

For illustration, FIG. 1 shows a plot trend of the measured temperature of an actual algae water slurry compared to measured ambient temperature during algal biomass cultivation. As shown, as ambient temperature rises and falls, so does the temperature of the algae slurry. These thermal fluctuations of the algae slurry can be detrimental to the cultivating algae cells therein, and further limit the particular strain(s) of algae cells that may be utilized and the location(s) of particular algae facilities. Indeed, temperatures greater than about 35° C. or 40° C. and less than about 10° C. can be lethal to many algae cells currently in use for producing algal biomass. In general, depending on the particular strain of algae, optimal algae temperatures may be in the range of about 25° C. to about 35° C., encompassing any value and subset therebetween, thus representing a relatively narrow band of optimal growth temperature. As shown in FIG. 1, for the particular algae slurry observed, the slurry temperature peaks over 35° C. on multiple days (i.e., Days 3-7). As provided above, temperatures that approach equal to or less than about 10° C. or equal to or greater than about 35° C. may negatively affect the growth of the algae cells, reducing the productivity of the algae facility and limiting the environments where such facilities may be located.

Whether using open or closed cultivation systems, traditional cultivation methods often control temperature fluctuations by utilizing power-driven processes (e.g., natural gas power plants) to heat and/or cool an algae water slurry, which are particularly expensive in terms of cost, energy consumption, and facility footprint requirements, especially given the large volumes of algae slurry that must be temperature controlled for large-scale algal biomass production.

The present disclosure provides systems and methods that utilize temperature-based, phase-transitioning (“TPT”) materials to passively control algae water slurry temperatures due to ambient environmental fluctuations during algae cultivation. The TPT material is used to form a thermal reservoir to maintain a specific temperature (or temperature range), selected for optimal algae growth (which may depend on the particular algae species, for example, as provided above), using latent heat of fusion to either absorb or release heat to an algae water slurry during cultivation thereof. The thermal reservoirs described herein may be beneficially integrated with a cultivation vessel. As described in greater detail below, the TPT material may be housed or otherwise contained within an enclosure (e.g., a chamber or liner) in contact with at least a portion of an algae water slurry within a cultivation vessel (open or closed) to provide passive temperature control. That is, the TPT material itself does not contact or otherwise transfer to an algae water slurry. When the ambient temperature of the algae water slurry exceeds the melting point of the TPT material, the TPT material melts and releases heat, which passes through the enclosure and is absorbed by a circulating algae water slurry. Conversely, when the ambient temperature of the algae water slurry reduces below the melting point of the TPT material, it freezes and releases heat, which is passed through the enclosure to the circulating algae water slurry.

The passive thermal control of algae water slurries using a TPT material as described here do not rely on moving, motorized, or electronic parts that often require maintenance or can otherwise hinder algae cultivation operations if failure occurs. Further, the aspects described herein do not require any artificial energy source or transportation of fluids throughout an algae facility to convey heat to a circulating algae slurry, which often requires substantial energy and other costs (e.g., larger facility footprint). Accordingly, the present disclosure allows the temperature of an algae water slurry to be at least partially controlled and maintained within a desired range while reducing associated energy consumption and other costs, including costs associated with potential compromise or loss of cultivated algae cells, while providing a single mechanism for both heating and cooling, thereby optimizing the productivity of an algae production facility.

As used herein, the term “thermal reservoir,” and grammatical variants thereof, refers to a reservoir (e.g., an enclosure) capable of absorbing or releasing thermal energy due to the presence of a TPT material. As described hereinbelow, examples of such thermal reservoirs may include a liner lining an open algae cultivation vessel or a chamber core or jacket in contact with a closed algae cultivation vessel.

As used herein, the term “integrated,” and grammatical variants thereof, with reference to at least a cultivation vessel and a thermal reservoir means that each are co-located and operate or function as a single assembly. For example, at least one portion of the cultivation vessel and/or the thermal reservoir are in thermal communication.

As used herein, the term “algae slurry,” “algae water slurry,” or “water slurry,” and grammatical variants thereof, refers to a flowable liquid comprising at least water, algae cells, and algae nutrient media (e.g., phosphorous, nitrogen, and optionally additional elemental nutrients), regardless of the confluency of the algae cell growth (and, thus, regardless of the viscosity of the slurry as a whole).

Algal sources for preparing the algae slurry include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In some examples, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species. Any combination of the aforementioned algae sources may additionally be used to prepare an algae slurry.

The water for use in preparing the algae slurry may be from any water source including, but not limited to, fresh water, brackish water, seawater, wastewater (treated or untreated), synthetic seawater (e.g., water with added salts), and any combination thereof.

The algae nutrient media for use in forming an algae slurry may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

As used herein, the term “cultivation vessel,” “vessel,” and grammatical variants thereof, refers to any of an open or closed algae cultivation system used for the growth of algal biomass, including bioreactors, photobioreactors, natural ponds, artificial ponds (e.g., raceway ponds), and the like.

A thermal reservoir comprising the TPT material for regulating or otherwise managing temperature of a circulating algae water slurry, as described herein, provides a low-cost solution (e.g., the TPT materials are relatively inexpensive) without undue operational complexity for controlling at least one aspect of the requirements for quality algae cultivation, thereby lending itself to scale-up operations using large-scale open and/or closed cultivation vessels. For example, to compete merely with U.S. diesel demand, a single algae biofuel facility would likely need to produce at least 10 thousand barrels per day (kbd), or even more (e.g., 20 kbd), to be viable, which is on par with current refinery facilities producing petroleum products. Accordingly, the total area of an open vessel system for true commercial algal biomass cultivation would need to be extremely large, requiring large vessels covering hundreds, or even thousands, of total surface area acreage. The passive algae water slurry temperature control systems and methods of the present disclosure are suited for relatively simple integration into such large-scale facility systems, as described herein in greater detail.

As provided hereinabove, the TPT material of the present disclosure melts when its melting point is exceeded (absorbing heat) and freezes when exposed to temperatures below its melting point (releasing heat). Selection of the TPT material, accordingly, is of critical importance for use in a thermal reservoir for temperature management of a circulating water slurry (e.g., within a liner or chamber as described hereinbelow). The TPT material(s) should be selected such that its melting point is within the high and low ambient temperature of the environment in which an algae vessel will be used and for the period of time the algae will be cultivated, typically about one day to one or two weeks, encompassing any value and subset therebetween. It is to be appreciated that such TPT material(s) will provide temperature control, although may not necessarily provide for a constant temperature, thereby eliminating or reducing high and low temperature spikes that can reduce the health and quality of the algae. For example, to prevent excursions above a critical algae cultivation temperature, the TPT may be selected such that it melts at or slightly below the critical temperature. In such instances, the TPT may never melt until needed to protect the algae on hot days. In another example, a TPT may be selected such that it fully melts at a critical temperature such that the heat absorbed by the TPT may diminish certain high temperatures experienced by a cultivating algae.

However, in one or more aspects, a thermal reservoir for maintaining a constant or substantially constant temperature (e.g., +/−1° C.) of a circulating algae water slurry (e.g., within a liner or chamber as described hereinbelow) may include a TPT material selected such that it does not fully melt or fully freeze. As such, while it is not fully melted it may continuously absorb heat from an algae water slurry and while it is not fully frozen it may continuously release heat to the algae water slurry. Accordingly, in one or more aspects of the present disclosure, the TPT material is selected to have a melting point that is close or equal to that of the mean ambient temperature experienced by an algae water slurry; that is, the mean ambient temperature of the environment in which the algae vessel that will contain the slurry is located. In certain examples, the selected TPT material may have a melting point that is within the range of +/−about 5° C. from the mean ambient temperature of the environment in which an algae vessel, encompassing any value and subset therebetween, will be used and for the period of time the algae will be cultivated, typically about one day to one or two weeks, encompassing any value and subset therebetween. In so selecting the TPT material, the thermal reservoir is balanced with daily heat release (and absorption).

In one or more aspects of the present disclosure, the selected TPT material comprises sodium sulfate decahydrate (H₂₀Na₂O₁₄S) (“SSD”). SSD has a melting temperature of approximately 32° C., which is in the range of optimal algae growth temperatures described above (i.e., about 25° C. to about 35° C.), and additionally has a large heat of fusion (i.e., heat absorbed by a unit mass of SSD at melting point) of approximately 250 kilojoule per kilogram (kJ/kg). Moreover, the melting temperature of SSD can be easily depressed by the addition (doping) of sodium chloride (NaCl) to easily account for seasonal fluctuations, as described below. SSD is further low-cost and widely available, thus making it particularly desirable as a TPT material for use in the methods and systems described herein. Other suitable TPT materials include, but are not limited to, paraffin waxes, methyl palmitate, 63 wt % trimetholethane in water solution, trimyristin, calcium chloride hexahydrate, copper nitrate hexahydrate, iron nitrate hexahydrate, hydroxylamine, lithium nitrate trihydrate, manganese nitrate hexahydrate, sodium carbonate heptahydrate, sodium carbonate decahydrate, and the like, and any combination thereof. The thermal reservoir may be filled with pure (only) TPT material or, in other instances, the TPT material may be intermixed with a liquid (e.g., a liquid surrounding solid TPT materials) which may displace air and improve heat transfer between the thermal reservoir and cultivating algae. Selection of the quantity of TPT material per unit volume of algae slurry may be based on a number of cost benefit optimizations, an example ratio is provided in the Example hereinbelow.

Mean ambient temperature at any environment fluctuates seasonally, as well as with specific weather phenomena. Accordingly, the present disclosure provides various methodologies for fine-tuning the thermal reservoirs described herein to account for such seasonal fluctuations. In one or more aspects, seasonal fluctuations may be accounted for by altering TPT material within the thermal reservoir to account for altering mean ambient temperature, supplementing the thermal reservoir with other heating and/or cooling systems, and/or distributing TPT material to account for any “spent” material (i.e., TPT material that is no longer absorbing or releasing heat).

Seasonal fluctuations may be accounted for by altering TPT material within the thermal reservoir to account for altering mean ambient temperature. That is, seasonal replacement or in situ adjustment of the particular TPT material (e.g., addition of NaCl to SSD) can be used to ensure that the thermal reservoir is as close or equal to the mean ambient temperature for year-round cultivation temperature control. This methodology matches the algae growth temperature to the mean ambient temperature, which in some instances may be different than the optimal algae growth temperature (e.g., 30° C.).

In one or more aspects, seasonal fluctuations may be accounted for by utilizing, in addition to the thermal reservoir, supplemental heating and/or cooling systems. These systems can be used to compensate for unwanted heat transfer due to, for example, maintaining a non-zero average temperature differential between a cultivating algae slurry and the environment, thereby permitting the cultivating algae slurry to remain at its optimal growth temperature. An advantage for combining the thermal reservoir and a supplemental heating and/or cooling system permits a reduction in the size, energy requirements, and other associated costs of such systems run alone because the thermal reservoir provides its own temperature moderation. As such, any supplemental heating and/or cooling system may be run at near steady state.

In one or more aspects, seasonal fluctuations may be abated by distributing “fresh” TPT material to replace “spent” TPT material. Spent TPT material includes TPT materials that are fully melted or fully frozen such that the TPT material is no longer providing heat absorption and/or release. For example, when the mean daily temperature has shifted out of a range that prevents the TPT material to fully reset. Centralized inventory of TPT material may be provided within an algae facility and fresh TPT material may be pulled from this inventory and delivered to one or more cultivation vessels comprising cultivating algae slurries and any spent TPT material is otherwise purged. In some instances, for example, melted TPT material may be replaced with frozen TPT material from a centralized inventory. The replacement and purging may be performed continuously or during turnarounds (new algae seedings) and may be partial replacement (e.g., separation based on liquid v. solid TPT material) or full replacement.

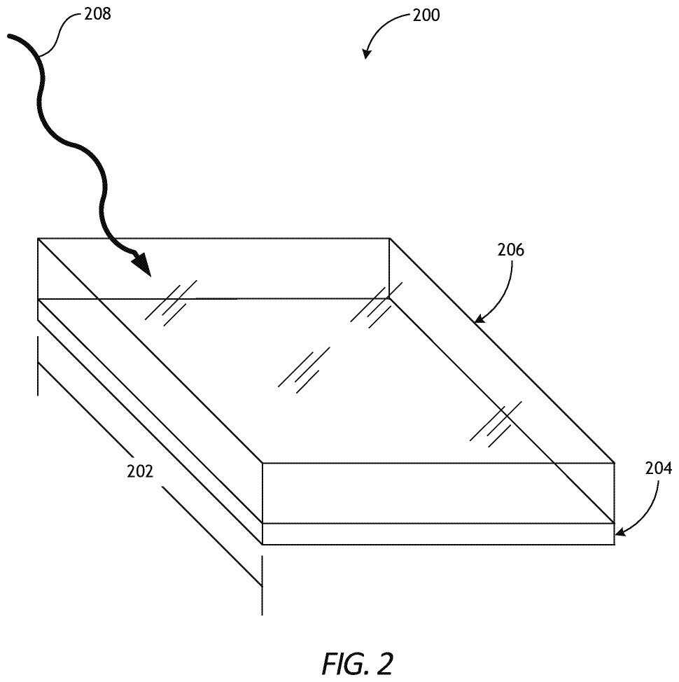

It is to be appreciated that the thermal reservoirs described herein may be employed or otherwise integrated with a cultivation vessel in various non-limiting configurations, in accordance with the aspects of the present disclosure. For illustrative purposes, FIGS. 2-4 provide examples of such configurations, for which the present disclosure is not limited.

Referring to FIG. 2, illustrated is a schematic isometric view of a portion of an integrated system 200 comprising an open cultivation vessel 202 and thermal reservoir 204. The thermal reservoir 204 is located at the base (i.e., interior or bottom surface of a circulation channel) of the open cultivation vessel 202. Other liners may or may not exist below the thermal reservoir 204; however thermal reservoir 204 is the uppermost liner such that circulating algae slurry 206 is in contact with thermal reservoir 204. Thermal reservoir 204 comprises one or more TPT material(s) (e.g., SSD). As incident light (e.g., sunlight) 208 contacts the cultivating algae slurry 206, it influences the temperature of the cultivating algae slurry 206. Depending on the fluctuation above or below the melting point of the TPT material within the thermal reservoir 204, the TPT material absorbs or releases heat to provide temperature control to the circulating algae slurry 206.

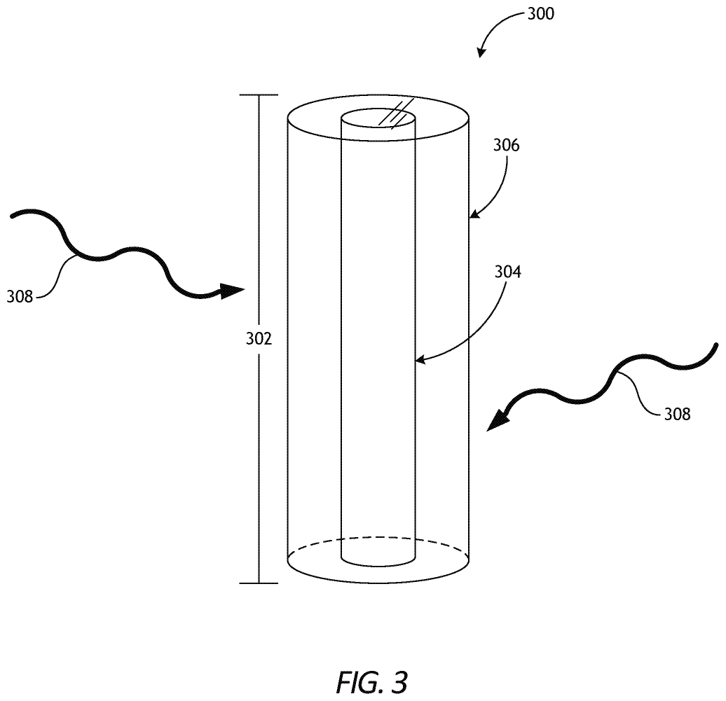

Referring now to FIG. 3, illustrated is a schematic side view of a portion of an integrated system 300 comprising a closed cultivation vessel 302 and thermal reservoir 304. As shown, closed cultivation vessel 302 may be in the form of a tubular photobioreactor. While the cultivation vessel 302 is shown as a vertical photobioreactor, it is to be appreciated that horizontal or skewed (e.g., slanted) photobioreactors may equally be used in accordance with the aspects of the present disclosure. The thermal reservoir 304 is located within closed cultivation vessel 302 as a core formed therein. As such, cultivating algae slurry 306 surrounds and is in contact with the thermal reservoir 304. Thermal reservoir 304 comprises one or more TPT material(s) (e.g., SSD). As incident light (e.g., sunlight) 308 contacts the cultivating algae slurry 306 (at any angle along tubular closed cultivation vessel 302), it influences the temperature of the cultivating algae slurry 306. Depending on the fluctuation above or below the melting point of the TPT material within the thermal reservoir 304, the TPT material absorbs or releases heat to provide temperature control to the circulating algae slurry 306.

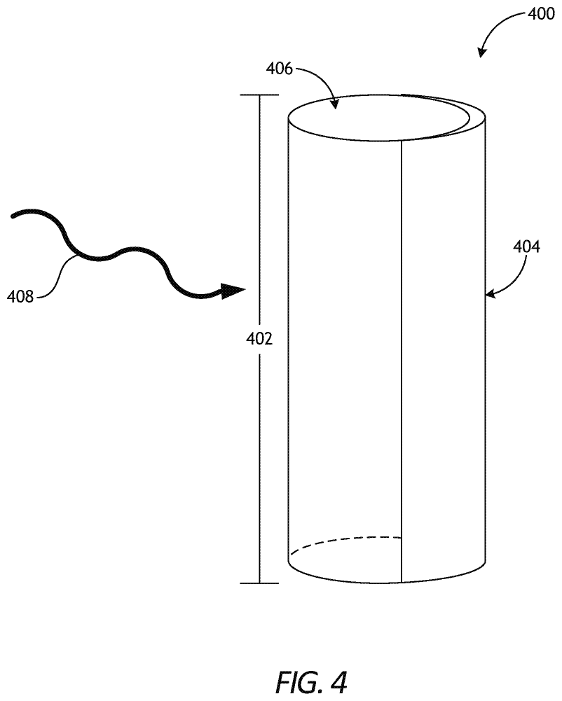

In other configurations, and referring now to FIG. 4, illustrated is a schematic side view of a portion of an integrated system 400 comprising a closed cultivation vessel 402 and thermal reservoir 404. As shown, and like FIG. 3, closed cultivation vessel 402 may be in the form of a tubular photobioreactor. While the cultivation vessel 402 is shown as a vertical photobioreactor, it is to be appreciated that horizontal or skewed (e.g., slanted) photobioreactors may equally be used in accordance with the aspects of the present disclosure. The thermal reservoir 404 is located outside of cultivation vessel 402 as a jacket formed thereover, and is jacketed over at least a portion of cultivation vessel 402, provided that incident light 408 is able to access the cultivating algae slurry 406. Moreover, thermal reservoir 404 may jacket the entirety of the cultivation vessel 402, wherein slots, holes, or other openings (that ensure containment of the TPT material within the thermal reservoir 404) may be disposed therein to allow incident light 408 to access the cultivating algae slurry 406.

Accordingly, cultivating algae slurry 406 is surrounded by and in contact with the thermal reservoir 404. Thermal reservoir 404 comprises one or more TPT material(s) (e.g., SSD). As incident light (e.g., sunlight) 408 contacts the cultivating algae slurry 406 (at any angle along tubular closed cultivation vessel 402 depending upon the configuration of thermal reservoir 404), it influences the temperature of the cultivating algae slurry 406. Depending on the fluctuation above or below the melting point of the TPT material within the thermal reservoir 404, the TPT material absorbs or releases heat to provide temperature control to the circulating algae slurry 406.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present disclosure. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more elements are presented herein. Not all features of a physical implementation are described or shown in this application for the sake of clarity. It is understood that in the development of a physical embodiment incorporating one or more elements of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

The present disclosure provides, among others, the following aspects, each of which may be considered as optionally including any alternate thereof

Clause 1: A system comprising: a cultivation vessel for containing an algae water slurry for cultivation; and a thermal reservoir integrated with the cultivation vessel for contact with and temperature control of the algae water slurry, the thermal reservoir comprising a temperature-based, phase-transitioning material.

Clause 2: The system of Clause 1, wherein the temperature-based, phase-transitioning material has a melting point in the range of +/−about 5° C. from a mean, environmental ambient temperature.

Clause 3: The system of Clause 1 or Clause 2, wherein the temperature-based, phase-transitioning material is selected from the group consisting of sodium sulfate decahydrate, paraffin waxes, methyl palmitate, 63 wt % trimetholethane in water solution, trimyristin, calcium chloride hexahydrate, copper nitrate hexahydrate, iron nitrate hexahydrate, hydroxylamine, lithium nitrate trihydrate, manganese nitrate hexahydrate, sodium carbonate heptahydrate, sodium carbonate decahydrate, and any combination thereof.

Clause 4: The system of any of the preceding Clauses, wherein the temperature-based, phase-transitioning material comprises sodium sulfate decahydrate.

Clause 5: The system of any of Clause 1 to Clause 4, wherein the cultivation vessel is an open cultivation vessel.

Clause 6: The system of any of Clause 1 to Clause 4, wherein the cultivation vessel is an open cultivation vessel and the thermal reservoir is in the form of a liner within the cultivation vessel.

Clause 7: The system of any of Clause 1 to Clause 4, wherein the cultivation vessel is a closed cultivation vessel.

Clause 8: The system of any of Clause 1 to Clause 4, wherein the cultivation vessel is a tubular, closed cultivation vessel and the thermal reservoir is in the form of a core within the tubular, closed cultivation vessel.

Clause 9: The system of any of Clause 1 to Clause 4, wherein the cultivation vessel is a tubular, closed cultivation vessel and the thermal reservoir is in the form of a jacket disposed over at least a portion of an outside of the tubular, closed cultivation vessel.

Clause 10: A method comprising: cultivating an algae water slurry within a cultivation vessel; and controlling a temperature of the algae water slurry using a thermal reservoir integrated with the cultivation vessel and in contact with the algae water slurry, the thermal reservoir comprising a temperature-based, phase-transitioning material.

Clause 11: The method of Clause 10, wherein the temperature-based, phase-transitioning material has a melting point in the range of +/−about 5° C. from a mean, environmental ambient temperature.

Clause 12: The method of Clause 10 or Clause 11, wherein the temperature-based, phase-transitioning material is selected from the group consisting of sodium sulfate decahydrate, paraffin waxes, methyl palmitate, 63 wt % trimetholethane in water solution, trimyristin, calcium chloride hexahydrate, copper nitrate hexahydrate, iron nitrate hexahydrate, hydroxylamine, lithium nitrate trihydrate, manganese nitrate hexahydrate, sodium carbonate heptahydrate, sodium carbonate decahydrate, and any combination thereof.

Clause 13: The method of any of Clause 10 to Clause 12, wherein the temperature-based, phase-transitioning material comprises sodium sulfate decahydrate.

Clause 14: The method of Clause 13, further comprising doping the thermal reservoir with sodium chloride to depress a melting temperature of the sodium sulfate decahydrate.

Clause 15: The method of any of Clause 10 to Clause 14, wherein the cultivation vessel is an open cultivation vessel.

Clause 16: The method of any of Clause 10 to Clause 14, wherein the cultivation vessel is an open cultivation vessel and the thermal reservoir is in the form of a liner within the cultivation vessel.

Clause 17: The method of any of Clause 10 to Clause 14, wherein the cultivation vessel is a closed cultivation vessel.

Clause 18: The method of any of Clause 10 to Clause 14, wherein the cultivation vessel is a tubular, closed cultivation vessel and the thermal reservoir is in the form of a core within the tubular, closed cultivation vessel.

Clause 19: The method of any of Clause 10 to Clause 14, wherein the cultivation vessel is a tubular, closed cultivation vessel and the thermal reservoir is in the form of a jacket disposed over at least a portion of an outside of the tubular, closed cultivation vessel.

To facilitate a better understanding of one or more aspects of the present disclosure, the following example is given. In no way should the following example be read to limit, or to define, the scope of the disclosure.

### Example

In this example, the feasibility of an integrated closed cultivation vessel comprising a core-type thermal reservoir was examined. Provided a core-type thermal reservoir of 5 inches in diameter within a 10 inch diameter photobioreactor (closed cultivation vessel), it was determined that the thermal reservoir (“TR”) would be capable of absorbing heat during a half day of operation of the photobioreactor (“PBR”). It is to be noted that in this Example, it was assumed that ambient temperature is higher than the melting point of the TPT material during the day by 10° C. Alternatively, if it was assumed that the ambient temperature dropped below the melting point of the TPT material, such as during the night, by 10° C., the TPT material would freeze in approximately 11 hours, thereby releasing heat.

The following assumptions were made: PBR length=6 feet; PBR diameter=10 inches; Core TR diameter=10 inches; TPT density=1 kilogram per liter (kg/L); TPT specific heat capacity=250 kJ/kg; Overall heat transfer coefficient for free convention=10 watts per square meter per degree Celsius (W/m²° C.); Representative temperature differential between algae slurry and environment=10° C. With these assumptions, the PBR external wall surface area can be calculated as being 1.46 m² ((π*10 iπ*2.54/100)*(6 ft*12*2.54/100)). The volume of the Core TR can be calculated as being 23.17 L ((π*5 in/2*2.54/100)²*(6 ft*12*2.54/100)*1000). The Core TR heat capacity (by virtue of the TPT material) can be calculated as being 5791.6 kJ (23.17 L*1 kg/L*250 kJ/kg). Additionally, the rate of heat transfer from the Core TR can be calculated as being 146 W (10 W/m²° C.*1.46 m²*10° C.). Accordingly, the time to complete chase transition of the TPT material within the Core TR (comprising the TPT material), assuming a small Biot number (dimensionless number used to quantify heat transfer), can be calculated as being 11 hours (˜half of a day) (5791.6 kJ/0.146 kJ/s).

Therefore, the aspects of the methods and systems presented herein are well adapted to attain the ends and advantages mentioned as well as those that are inherent therein. The particular examples and configurations disclosed above are illustrative only, as the present disclosure may be modified and practiced in different but equivalent manners apparent to those skilled in the art to having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present disclosure. The aspects illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

What is claimed is:

1. A system comprising:
a cultivation vessel for containing an algae water slurry for cultivation; and
a thermal reservoir integrated with the cultivation vessel for contact with and temperature control of the algae water slurry, the thermal reservoir comprising a temperature-based, phase-transitioning material.

2. The system of claim 1, wherein the temperature-based, phase-transitioning material has a melting point in the range of +/−about 5° C. from a mean, environmental ambient temperature.

3. The system of claim 1, wherein the temperature-based, phase-transitioning material is selected from the group consisting of sodium sulfate decahydrate, paraffin waxes, methyl palmitate, 63 wt % trimetholethane in water solution, trimyristin, calcium chloride hexahydrate, copper nitrate hexahydrate, iron nitrate hexahydrate, hydroxylamine, lithium nitrate trihydrate, manganese nitrate hexahydrate, sodium carbonate heptahydrate, sodium carbonate decahydrate, and any combination thereof.

4. The system of claim 1, wherein the temperature-based, phase-transitioning material comprises sodium sulfate decahydrate.

5. The system of claim 1, wherein the cultivation vessel is an open cultivation vessel.

6. The system of claim 1, wherein the cultivation vessel is an open cultivation vessel and the thermal reservoir is in the form of a liner within the cultivation vessel.

7. The system of claim 1, wherein the cultivation vessel is a closed cultivation vessel.

8. The system of claim 1, wherein the cultivation vessel is a tubular, closed cultivation vessel and the thermal reservoir is in the form of a core within the tubular, closed cultivation vessel.

9. The system of claim 1, wherein the cultivation vessel is a tubular, closed cultivation vessel and the thermal reservoir is in the form of a jacket disposed over at least a portion of an outside of the tubular, closed cultivation vessel.

10. A method comprising:
cultivating an algae water slurry within a cultivation vessel; and
controlling a temperature of the algae water slurry using a thermal reservoir integrated with the cultivation vessel and in contact with the algae water slurry, the thermal reservoir comprising a temperature-based, phase-transitioning material.

11. The method of claim 10, wherein the temperature-based, phase-transitioning material has a melting point in the range of +/−about 5° C. from a mean, environmental ambient temperature.

12. The method of claim 10, wherein the temperature-based, phase-transitioning material is selected from the group consisting of sodium sulfate decahydrate, paraffin waxes, methyl palmitate, 63 wt % trimetholethane in water solution, trimyristin, calcium chloride hexahydrate, copper nitrate hexahydrate, iron nitrate hexahydrate, hydroxylamine, lithium nitrate trihydrate, manganese nitrate hexahydrate, sodium carbonate heptahydrate, sodium carbonate decahydrate, and any combination thereof.

13. The method of claim 10, wherein the temperature-based, phase-transitioning material comprises sodium sulfate decahydrate.

14. The method of claim 13, further comprising doping the thermal reservoir with sodium chloride to depress a melting temperature of the sodium sulfate decahydrate.

15. The method of claim 10, wherein the cultivation vessel is an open cultivation vessel.

16. The method of claim 10, wherein the cultivation vessel is an open cultivation vessel and the thermal reservoir is in the form of a liner within the cultivation vessel.

17. The method of claim 10, wherein the cultivation vessel is a closed cultivation vessel.

18. The system of claim 10, wherein the cultivation vessel is a tubular, closed cultivation vessel and the thermal reservoir is in the form of a core within the tubular, closed cultivation vessel.

19. The method of claim 10, wherein the cultivation vessel is a tubular, closed cultivation vessel and the thermal reservoir is in the form of a jacket disposed over at least a portion of an outside of the tubular, closed cultivation vessel.
